(19) United States
(12) Patent Application Publication
(10) Pub. No.: US 2019/0083547 A1
(43) Pub. Date: Mar. 21, 2019

(54) TRANSDERMAL DELIVERY OF AMNION TISSUE PREPARATIONS

(71) Applicant: Gary M. Petrucci, Long Lake, MN (US)

(72) Inventor: Gary M. Petrucci, Long Lake, MN (US)

(21) Appl. No.: 15/708,425

(22) Filed: Sep. 19, 2017

(51) Int. Cl. A61K 35/50; A61K 9/00; A61K 35/28; A61N 1/32

(52) U.S. Cl. CPC A61K 35/50; A61K 9/0014; A61K 9/0009; A61N 1/328; A61K 35/28; A61N 1/325; A61K 9/0021

(57) ABSTRACT

This document relates to methods and materials for transdermal delivery of a composition including an amnion tissue preparation. For example, methods and materials for transdermal delivery of an amnion tissue preparation to treat wounds and/or skin disorders are provided.

### BACKGROUND

### 1. Technical Field

This document relates to methods and materials for transdermal delivery of a composition including an amnion tissue preparation. For example, transdermal delivery of a composition including an amnion tissue preparation can be used to treat wounds and/or skin disorders.

### 2. Background Information

Skin, one of the most extensive and readily accessible organs of the human body, has a multifunctional role. One of the most important functions is its ability to act as a protective barrier against foreign material such as chemicals and microbes, and against the loss of excessive endogenous material such as water.

### SUMMARY

This document provides compositions that include an amnion tissue preparation and, optionally, a stem cell preparation. Such compositions can be formulated for transdermal delivery. This document also provides methods for transdermally administering a composition that includes an amnion tissue preparation and, optionally, a stem cell preparation. For example, a composition that includes an amnion tissue preparation and, optionally, a stem cell preparation can be administered transdermally as described herein to treat wounds (e.g., a scar) and/or skin disorders (e.g., psoriasis).

In general, one aspect of this document features a method for healing a wound in a mammal. The method includes, or consists essentially of, transdermally administering a composition including an amnion tissue preparation lacking viable cells to a mammal, where the size of the wound is reduced. The mammal can be a human. The wound can be an abrasion, an incision, a laceration, a puncture, an avulsion, or a burn. The wound can be a slow healing wound. The wound can be a scar. The size of the wound can be reduced by at least 5%. The transdermal administering can include iontophoresis. The iontophoresis can include from about 0.1 mA/cm² to about 1.0 mA/cm². The transdermal administering can include microinjection. The microinjection can include using a microneedle array. The transdermal administering can include a transdermal delivery device. The transdermal delivery device can be an adhesive patch. The amnion tissue preparation can include an amnion tissue preparation prepared from about 1 mg to about 10 g of amnion tissue. The composition also can include a stem cell preparation having viable cells, where the stem cell preparation includes from about 0.3 million to about 3 million stem cells. The amnion tissue preparation can be a human amnion tissue preparation. The stem cell preparation can be a human mesenchymal stem cell preparation.

In another aspect, this document features a method for treating a skin disorder in a mammal. The method includes, or consists essentially of, transdermally administering a composition including an amnion tissue preparation lacking viable cells to the mammal, where a symptom of the skin disorder is reduced. The mammal can be a human. The skin disorder can be eczema, psoriasis, rosacea, seborrheic dermatitis, acne, cold sores, shingles, basal cell carcinoma, squamous cell carcinoma, melanoma, actinic keratosis, vitiligo, melasma, or lentigenes. The skin disorder can be psoriasis. The symptom can be eliminated.

The transdermal administering can include iontophoresis. The method iontophoresis can include from about 0.1 mA/cm² to about 1.0 mA/cm². The transdermal administering can include microinjection. The microinjection can include using a microneedle array. The transdermal administering can include a transdermal delivery device. The transdermal delivery device can be an adhesive patch. The amnion tissue preparation can include an amnion tissue preparation prepared from about 1 mg to about 10 g of amnion tissue. The composition also can include a stem cell preparation having viable cells, where the stem cell preparation includes from about 0.3 million to about 3 million stem cells. The amnion tissue preparation can be a human amnion tissue preparation. The stem cell preparation can be a human mesenchymal stem cell preparation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document provides methods and materials for treating wounds (e.g., scars) and/or skin disorders (e.g., psoriasis). For example, one or more wounds and/or skin disorders can be treated by administering (e.g., via transdermal delivery) an effective amount of a composition provided herein (e.g., a composition that includes an amnion tissue preparation and, optionally, a stem cell preparation). In some cases, the methods and materials described herein can be used to enhance wound healing. For example, transdermal delivery of a composition that includes an amnion tissue preparation described herein and, optionally, a stem cell preparation described herein can be used to reduce the size of a wound. In some cases, the methods and materials described herein can be used to treat a skin disorder. For example, transdermal delivery of a composition that includes an amnion tissue preparation described herein and, optionally, a stem cell preparation described herein can be used to reduce the severity of a skin disorder and/or to reduce one or more symptoms of a skin disorder.

In some cases, the methods and materials for treating mammals (e.g., humans) having one or more wounds and/or a skin disorder can include identifying the mammal as having one or more wounds and/or a skin disorder. Any appropriate method can be used to identify a mammal as having one or more wounds and/or as having a skin disorder. An example of a method that can be used to identify a mammal as having a wound includes, without limitation, physical examination. Examples of methods that can be used to identify a mammal as having a skin disorder include, without limitation, physical examination, prick tests, laboratory tests, and imaging analyses. Once identified as having one or more wounds and/or a skin disorder, the mammal can be transdermally administered or instructed to transdermally self-administer a composition described herein (e.g., a composition that includes an amnion tissue preparation and, optionally, a stem cell preparation).

Any type of mammal having one or more wounds and/or a skin disorder can be treated using the methods and materials described herein. Examples of mammals that can be treated with a composition described herein (e.g., a composition that includes an amnion tissue preparation and, optionally, a stem cell preparation) include, without limitation, primates (e.g., humans and non-human primates such as chimpanzees, baboons, or monkeys), dogs, cats, horses, cows, pigs, sheep, rabbits, mice, rats, guinea pigs, gerbils, hamsters, horses, goats, and mammalian zoo animals (e.g., a lion, a tiger, or a leopard). In some cases, the mammal can a human. For example, a human having one or more wounds and/or a skin disorder can be treated by administering (e.g., via transdermal delivery) a composition that includes an amnion tissue preparation and, optionally, a stem cell preparation.

Any appropriate type of wound can be treated using the methods and materials described herein. The term “wound” as used herein refers to any skin injury in which damage (e.g., abrasion, incision, laceration, puncture, avulsion, or burn) has occurred to skin. A wound can affect any part of the skin including, without limitation, the epidermis (e.g., the stratum corneum), the dermis, and/or the hypodermis. A wound can be an open wound or a closed wound. A wound can be a chronic (e.g., a slow healing) wound. A wound can be a scar. A wound can be located on any part of the body (e.g., arm, leg, foot, hand, belly, chest, and back.

Any appropriate type of skin disorder can be treated using the methods and materials described herein. The term “skin disorder” as used herein refers to any condition that affects (e.g., directly or indirectly) the function of a dermal tissue. Skin disorders can include, without limitation, eczema, psoriasis, rosacea, seborrheic dermatitis, acne, disorders caused by herpes simplex infection (e.g., cold sores), disorders caused by herpes zoster infection (e.g., shingles), skin cancers (e.g., basal cell carcinoma, squamous cell carcinoma and melanoma), sun damage (e.g., actinic keratosis), and pigmentary disorders (e.g., vitiligo, melasma, and lentigenes).

In some cases, the methods and materials described herein can be used to treat other diseases and/or disorders including, but not limited to, lung disease, heart disease, neurological disorders, neurological injuries, muscular disorders, and muscular injuries.

A composition provided herein can include any appropriate amnion tissue preparation. The term “amnion tissue preparation” as used herein refers to a preparation of amnion tissue or amnion material. The term “dried amnion tissue preparation” as used herein refers to a preparation of amnion tissue or amnion material that is dried to have a water content that is less than about 8 percent (e.g., less than about 7 percent, less than about 6 percent, less than about 5 percent, less than about 4 percent, less than about 3 percent, less than about 2 percent, or less than about 1 percent). In some cases, a dried amnion tissue preparation can have a water content that is between about 0.1 percent and about 8 percent (e.g., between about 0.5 percent and about 8 percent, between about 1 percent and about 8 percent, between about 0.1 percent and about 5 percent, between about 0.1 percent and about 4 percent, between about 0.1 percent and about 3 percent, between about 0.5 percent and about 5 percent, or between about 1 percent and about 4 percent). An amnion tissue preparation can be dried using any appropriate technique such as micronization, vacuum drying, spray drying, freeze drying, or combinations thereof. In some cases, an amnion tissue preparation can be dried as described elsewhere (e.g., U.S. Pat. No. 5,656,498). In some cases, an amnion tissue preparation can be a liquid preparation (e.g., solution or suspension) that is prepared from a dried amnion tissue preparation.

An amnion tissue preparation (e.g., a dried amnion preparation) described herein can have any appropriate particle size. For example, a dried amnion tissue preparation can have a particle size ranging from about 0.1 μm to about 25 μm (e.g., from about 0.5 μm to about 25 μm, from about 0.75 μm to about 25 μm, from about 1 μm to about 25 μm, from about 0.1 μm to about 15 μm, from about 0.1 μm to about 10 μm, from about 0.1 μm to about 7.5 μm, from about 0.1 μm to about 5 μm, from about 0.75 μm to about 7.5 μm, or from about 1 μm, to about 5 μm).

An amnion tissue preparation (e.g., a dried amnion preparation) described herein can contain viable cells, non-viable cells, or a combination thereof. For example, an amnion tissue preparation or a dried amnion tissue preparation can be a preparation of amnion tissue or amnion material having viable cells. In some cases, an amnion tissue preparation can be a solution or suspension of amnion tissue or amnion material having viable cells. In some cases, an amnion tissue preparation or a dried amnion tissue preparation can be a preparation of amnion tissue or amnion material where all the cells were removed, killed, or lysed such that the amnion tissue preparation or the dried amnion tissue preparation lacks viable cells. In some cases, an amnion tissue preparation or a dried amnion tissue preparation can be a preparation of amnion tissue or amnion material that was exposed to one or more physical and/or chemical treatments that killed, fixed, or lysed the cells of the amnion tissue or amnion material such that the amnion tissue preparation or the dried amnion tissue preparation lacks viable cells. For example, temperature (e.g., rapid freezing or rapid freezing-thawing), force and pressure, and/or electrical disruption can be used to kill or lyse cells within amnion tissue or amnion material to produce an amnion tissue preparation or a dried amnion tissue preparation that lacks viable cells.

In some cases, amnion tissue or amnion material can be obtained and then treated in a manner designed to lyse all the cells within the amnion tissue or amnion material. In these cases, the resulting material (e.g., matrix material and cellular remnants from lysed cells) can be used as an amnion tissue preparation that lacks viable cells or dried to form a dried amnion tissue preparation that lacks viable cells.

In some cases, an amnion tissue preparation (e.g., a dried amnion tissue preparation) can be prepared from human amnion tissue. For example, human amnion tissue can be harvested, processed to maintain cell viability with or without removing blood, and used as an amnion tissue preparation or dried to form a dried amnion tissue preparation.

In some cases, human amnion tissue can be processed to remove blood prior to being used as an amnion tissue preparation or prior to being dried to form a dried amnion tissue preparation. In some cases, human amnion tissue can be processed without removing cells or blood prior to forming an amnion tissue preparation or a dried amnion tissue preparation.

An example of an amnion tissue preparation includes, without limitation, a human amnion tissue preparation that includes viable cells. In some cases, an amnion tissue preparation can be obtained from MiMedX® or a tissue bank (e.g., a human tissue bank).

A composition provided herein can include any appropriate stem cell preparation. The term “stem cell preparation” as used herein refers to a preparation of stem cells or stem cell material. In some cases, a stem cell preparation can be a liquid preparation (e.g., solution or suspension).

A stem cell preparation can contain viable stem cells, non-viable stem cells, or a combination thereof. For example, a stem cell preparation can be a preparation of viable stem cells. In some cases, a stem cell preparation can be a solution or suspension of viable stem cells.

In some cases, a stem cell preparation can be a preparation of stem cell or stem cell material where all the stem cells were killed, fixed, or lysed such that the stem cell preparation lacks viable stem cells. In some cases, a stem cell preparation can be a preparation of stem cells or stem cell material that was exposed to one or more physical and/or chemical treatments that killed, fixed, or lysed the stem cells such that the stem cell preparation lacks viable stem cells. For example, temperature (e.g., rapid freezing or rapid freezing-thawing), force and pressure, and/or electrical disruption can be used to kill or lyse stem cells to produce a stem cell preparation that lacks viable stem cells.

In some cases, a stem cell culture can be obtained and then used as a stem cell preparation in a manner that maintains stem cell viability.

Examples of stem cell preparations include, without limitation, a mesenchymal stem cell (MSC) preparation (e.g., a MSC preparation obtained from fat tissue or bone marrow), a neural stem cell (NSC) preparation (e.g., a NSC preparation obtained from a brain tissue such as striatum), an umbilical cord blood stem cell preparation, an embryonic stem cell preparation, and a human induced pluripotent stem cell preparation.

In some cases, stem cell preparations can be prepared from cultures of stem cells. For example, a culture containing from about 25 million to about 25 billion (e.g., from about 25 million to about 20 billion, from about 25 million to about 15 billion, from about 25 million to about 10 billion, from about 25 million to about 5 billion, from about 25 million to about 1 billion, from about 50 million to about 25 billion, from about 75 million to about 25 billion, from about 100 million to about 25 billion, from about 150 million to about 25 billion, from about 250 million to about 25 billion, from about 500 million to about 25 billion, from about 50 million to about 20 billion, from about 75 million to about 15 billion, from about 100 million to about 10 billion, from about 150 million to about 5 billion, from about 250 million to about 2 billion, or from about 500 million to about 1 billion) stem cells can be used to make a stem cell preparation.

In some cases, a stem cell preparation can be obtained commercially from a variety of suppliers such as Stemedica Cell Technologies, Inc.

In some cases, an amnion tissue preparation and a stem cell preparation can be formulated into a single solution or suspension for administration to a mammal. For example, a dried amnion tissue preparation can be reconstituted into a solution and a stem cell preparation can be added to that solution to form a single solution or suspension having both an amnion tissue preparation and a stem cell preparation.

A composition described herein (e.g., a composition that includes an amnion tissue preparation described herein and, optionally, a stem cell preparation described herein) can be formulated into a pharmaceutically acceptable composition for administration (e.g., via transdermal delivery) to a mammal having one or more wounds and/or a skin disorder. For example, a composition that includes an amnion tissue preparation described herein and, optionally, a stem cell preparation described herein can be formulated together with one or more pharmaceutically acceptable carriers (additives) and/or diluents for transdermal delivery to a human having one or more wounds and/or a skin disorder.

A composition described herein (e.g., a composition that includes an amnion tissue preparation described herein and, optionally, a stem cell preparation described herein) can be in any appropriate form including, without limitation, solid, liquid, gel, suspension, powder, and granules. For example, a composition can be in any appropriate form for use with transdermal delivery. In some cases, a composition described herein can include one or more delivery vesicles (e.g., non-ionic based surfactant vesicles, and lipid vesicles). In some cases, a composition described herein can be an ionic composition (e.g., cationic or anionic). In some cases, a composition described herein can include one or more chemical penetration enhancers (e.g., alcohols, sulphoxides, azones, pyrrolidones, essential oils, terpenes and terpenoids, fatty acids, water, and/or urea).

A composition described herein (e.g., a composition that includes an amnion tissue preparation described herein and, optionally, a stem cell preparation described herein) can have any appropriate particle size. A composition can include nanoparticles and/or microparticles. For example, a composition that includes an amnion tissue preparation described herein and, optionally, a stem cell preparation described herein can have a particle size suitable for transdermal delivery. In some cases, a composition can have a particle size ranging from about 0.1 μm to about 100 μm (e.g., from about 0.1 μm to about 75 μm, from about 0.1 μm to about 50 μm, from about 0.1 μm to about 25 μm, from about 0.5 μm to about 100 μm, from about 1 μm to about 100 μm, from about 5 μm to about 100 μm, from about 10 μm to about 100 μm, from about 25 μm to about 100 μm, from about 50 μm to about 100 μm, from about 0.5 μm to about 75 μm, from about 1 μm to about 50 μm, from about 5 μm to about 40 μm, from about 8 μm to about 25 μm, or from about 10 μm to about 20 μm).

A composition described herein (e.g., a composition that includes an amnion tissue preparation described herein and, optionally, a stem cell preparation described herein) also can include one or more therapeutic agents, one or more immunosuppressant agents (e.g., corticosteroids (e.g., glucocorticoids), cytostatics, antibodies, calcineurin inhibitors, and interferons), one or more anti-inflammatory agents (e.g., non-steroidal anti-inflammatory drugs, dexamethasone or other type of glucocorticoid steroids), one or more growth factors (e.g., platelet derived growth factor (PDGF), epithelial growth factor (EGF), fibroblast growth factor-2 (FGF2), or stem cell factor (SCF)), and/or one or more antimicrobial agents (e.g., antibiotics such as kanamycin, neomycin, streptomycin, or gentamicin, or an antifungal agent).

As described herein, one or more wounds and/or skin disorders can be treated by administering (e.g., via transdermal delivery) an effective amount of a composition described herein (e.g., a composition that includes an amnion tissue preparation and, optionally, a stem cell preparation described herein). In some cases, effective amounts of compositions described herein can be determined by a physician, taking into account various factors such as overall health status, body weight, sex, diet, time and route of administration, other medications, and any other relevant clinical factors. As used herein, an “effective amount” or “therapeutically effective amount” of a composition provided herein is the amount that is sufficient to provide a beneficial effect to the mammal to which the composition or preparations are administered. The effective amount can be the amount effective to achieve a more rapid recovery, an improvement in the quality of life, reduced wound size, and/or the reduction or elimination of one or more symptoms associated with a mammal's wound and/or skin disorder.

A composition described herein (e.g., a composition that includes an amnion tissue preparation described herein and, optionally, a stem cell preparation described herein) can be administered using any appropriate technique. In some cases, an amnion tissue preparation described herein can be administered by transdermal delivery. For example, a composition containing an amnion tissue preparation and, optionally, a stem cell preparation described herein can be administered transdermally into the skin near the site of a wound and/or the skin of a mammal having a skin disorder. Transdermal delivery can be passive (e.g., chemical) or active (e.g., physical).

A composition described herein (e.g., a composition that includes an amnion tissue preparation described herein and, optionally, a stem cell preparation described herein) can be administered transdermally using any appropriate method of transdermal delivery. Methods of transdermal delivery that can be used to administer a composition described herein include, without limitation, electrical delivery (e.g., iontophoresis at about 0.1 mA/cm² to about 1.0 mA/cm² and electroporation at about 50 V to about 500 V), mechanical delivery (e.g., microneedle), ultrasound based delivery (e.g., sonophoresis and phonophoresis at frequencies of about 20 kHz to about 16 MHz), thermal ablation delivery (e.g., laser ablation and radiofrequency ablation at about 100 kHz to about 500 kHz), and injection delivery (e.g., microinjection, liquid injection, or dry injection).

In some cases, transdermal delivery methods can include administration (e.g., topical application) of a composition described herein (e.g., a composition that includes an amnion tissue preparation described herein and, optionally, a stem cell preparation described herein).

In some cases, transdermal delivery methods can include using a transdermal delivery device for administration (e.g., topical application) of a composition described herein (e.g., a composition that includes an amnion tissue preparation described herein and, optionally, a stem cell preparation described herein). Examples of transdermal delivery devices include, without limitation, ultrasound devices, iontophoresis devices (e.g., iontophoretic delivery systems such as iontophoresis patches), jet injectors (e.g., liquid injectors and dry (e.g., powder) injectors), and microneedle arrays (e.g., microneedle array patches). In some cases, a transdermal delivery device can include a patch (e.g., an adhesive patch). In some cases, a transdermal delivery device can be a needle-free device. In some cases, a transdermal delivery device can include one or more (e.g., 4 or more, 9 or more, 12 or more, 16 or more, 25 or more, 36 or more, 49 or more, 64 or more, 81 or more, or 100 or more) microneedles. A microneedle can be a dissolving microneedle or a biodegradable microneedle. A microneedle can be a silicon microneedle. A microneedle can be less than about 2000 μm in size (e.g., less than about 1000 μm, less than about 750 μm, less than about 500 μm, less than about 250 μm, less than about 200 μm, less than about 175 μm, less than about 150 μm, less than about 125 μm, or less than about 100 μm in size). For example, a microneedle can have a height from about 25 μm to about 2000 μm (e.g., from about 50 μm to about 2000 μm, from about 100 μm to about 2000 μm, from about 200 μm to about 2000 μm, from about 300 μm to about 2000 μm, from about 400 μm to about 2000 μm, from about 500 μm to about 2000 μm, from about 750 μm to about 2000 μm, from about 1000 μm to about 2000 μm, from about 25 μm to about 1500 μm, from about 25 μm to about 1250 μm, from about 25 μm to about 1000 μm, from about 25 μm to about 7500 μm, from about 25 μm to about 500 μm, or from about 25 μm to about 250 μm). For example, a microneedle can have a base width from about 50 μm to about 250 μm (e.g., from about 50 μm to about 225 μm, from about 50 μm to about 200 μm, from about 50 μm to about 175 μm, from about 50 μm to about 150 μm, from about 50 μm to about 125 μm, from about 50 μm to about 100 μm, from about 50 μm to about 75 μm, from about 75 μm to about 250 μm, from about 100 μm to about 250 μm, from about 125 μm to about 250 μm, from about 150 μm to about 250 μm, from about 200 μm to about 250 μm, from about 75 μm to about 225 μm, or from about 100 μm to about 200 μm). For example, a microneedle can have a tip diameter from about 1 μm to about 25 μm (e.g., from about 1 μm to about 22 μm, from about 1 μm to about 20 μm, from about 1 μm to about 17 μm, from about 1 μm to about 15 μm, from about 1 μm to about 12 μm, from about 1 μm to about 10 μm, from about 3 μm to about 25 μm, from about 5 μm to about 25 μm, from about 8 μm to about 25 μm, from about 10 μm to about 25 μm, from about 12 μm to about 25 μm, from about 15 μm to about 25 μm, from about 3 μm to about 22 μm, from about 5 μm to about 20 μm, or from about 8 μm to about 15 μm).

Transdermal delivery of a composition described herein (e.g., a composition that includes an amnion tissue preparation described herein and, optionally, a stem cell preparation described herein) can result in local exposure to the composition, systemic exposure to the composition, or a combination thereof.

A composition described herein (e.g., a composition that includes an amnion tissue preparation described herein and, optionally, a stem cell preparation described herein) can be formulated for a particular transdermal delivery method. For example, when the transdermal delivery method is an electrical transdermal delivery method, the composition can be an ionic composition. Methods of transdermal delivery can be as described elsewhere (see, e.g., Alkilani et al., Pharmaceutics, 7:438-470 (2015); Brown et al., Drug Delivery, 13:175-187 (2006); Kalluri et al., AAPS PharmSciTech, 12:431-441 (2011); and Kumar et al., Trop. J. Pharm. Res., 6:633-644 (2007)).

In some cases, transdermal delivery methods described herein also can include, prior to administration of a composition described herein (e.g., a composition that includes an amnion tissue preparation described herein and, optionally, a stem cell preparation described herein), preparation of the site of administration (e.g., the skin near a wound and/or the skin of a mammal having a skin disorder). Examples of ways that skin can be prepared for transdermal delivery include, without limitation, cleaning, hair removal (e.g., mechanical hair removal such as shaving, plucking, tweezing, and waxing, or chemical hair removal such as using depilatory creams), and applying one or more penetration enhancers (e.g., alcohols, sulphoxides, azones, pyrrolidones, essential oils, terpenes and terpenoids, fatty acids, water, and/or urea).

The methods described herein can include delivering to a mammal (e.g., a human) a composition described herein (e.g., a composition that includes an amnion tissue preparation described herein and, optionally, a stem cell preparation described herein) that is made with from about 0.01 mg to about 10 g (e.g., from about 0.01 mg to about 10 g, from about 0.1 mg to about 10 g, from about 1 mg to about 10 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 100 mg to about 1 g, or from about 1 g to about 5 g) of amnion tissue per ml and/or per mg.

In cases where a composition described herein (e.g., a composition that includes an amnion tissue preparation described herein and, optionally, a stem cell preparation described herein) includes a stem cell preparation, the methods described herein can include delivering to a mammal (e.g., a human) a stem cell preparation (e.g., a stem cell preparation having viable stem cells) that is made from about 0.1 million to about 3 million (e.g., from about 0.3 million to about 3 million, from about 0.5 million to about 3 million, from about 0.75 million to about 3 million, from about 1 million to about 3 million, from about 1.5 million to about 3 million, from about 0.3 million to about 2.5 million, from about 0.3 million to about 2.0 million, from about 0.3 million to about 1.5 million, from about 0.3 million to about 1.0 million, from about 0.5 million to about 2.5 million, from about 0.75 million to about 2.0 million, from about 0.8 million to about 1.5 million) stem cells.

In some cases, a composition described herein (e.g., a composition that includes an amnion tissue preparation described herein and, optionally, a stem cell preparation described herein) can be administered to a mammal (e.g., by transdermal delivery) only once. In some cases, multiple (e.g., two, three, four, five, six, seven, eight, nine, 10, 11, 12, 13, 14, 15, or 20 or more) administrations can be used. For example, multiple administrations of a composition described herein can be made over the course of several (e.g., two, three, four, five, six, seven, eight, nine, 10, 14, 21, 28, or 31 or more) consecutive days (e.g., one delivery each day for seven days or one delivery every other day for seven days). In some cases, a composition described herein can be administered from about once a week to about once per year (e.g., once every month or once every other month). In some cases, a composition described herein can be administered to a mammal for several months (e.g., one delivery per month for six months, or one delivery per week for two months).

A composition described herein (e.g., a composition that includes an amnion tissue preparation described herein and, optionally, a stem cell preparation described herein) can be administered (e.g., by transdermal delivery) to a mammal at various time points after diagnosis with a wound and/or a skin disorder. For example, a composition containing an amnion tissue preparation described herein and, optionally, a stem cell preparation described herein can be administered by transdermal delivery immediately following diagnosis with a wound and/or a skin disorder. In some cases, a composition containing an amnion tissue preparation described herein and, optionally, a stem cell preparation described herein can be administered to a mammal less than 10 (e.g., 9, 8, 7, 6, 5, 4, 3, 2, or 1) days after diagnosis with a wound and/or a skin disorder.

A composition described herein (e.g., a composition that includes an amnion tissue preparation described herein and, optionally, a stem cell preparation described herein) can be administered to a mammal as a combination therapy with another treatment used to treat a wound and/or a skin disorder. For example, a combination therapy can include administering to the mammal (e.g., a human) a composition described herein and one or more additional agents that provide a therapeutic effect to the mammal who has a wound and/or skin disorder. In some cases, the composition and the one or more additional agents can be administered at the same time. In some cases, the composition can be administered first, and the one or more additional agents administered second, or vice versa.

In some cases, the methods described herein can include monitoring one or more wounds and/or a skin disorder in the mammal (e.g., to determine the efficacy of the treatment). Any appropriate method can be used to monitor the wound and/or the skin disorder. For example, a wound can be monitored to determine if the wound is healing. In some cases, wound healing can be defined as reducing the size of the wound by at least 5% (e.g., at least 10%, at least 15%, at least 20%, at least 25%, at least 30%, at least 40%, at least 50%, at least 55%, at least 60%, at least 65% or more). For example, a skin disorder can be monitored to determine if the severity of the skin disorder and/or one or more symptoms of the skin disorder are being reduced. In some cases, one or more symptoms of the skin disorder can be eliminated.

A composition described herein (e.g., a composition that includes an amnion tissue preparation described herein and, optionally, a stem cell preparation described herein) can be combined with packaging material and sold as a kit. The packaging material included in a kit typically contains instructions or a label describing how the composition can be administered (e.g., via transdermal delivery) into the skin near a wound and/or into the skin of a mammal having a skin disorder. A kit also can include a unit dose device. The term “unit dose device” refers to a transdermal delivery device that administers a single dose of a composition described herein to a user (e.g., a human). Typically, a unit dose device contains a single container that holds or contains a pharmaceutically acceptable composition that includes an amnion tissue preparation described herein and, optionally, a stem cell preparation described herein.

The invention will be further described in the following examples, which do not limit the scope of the invention described in the claims.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for healing a wound in a mammal, wherein said method comprises:
transdermally administering a composition comprising an amnion tissue preparation lacking viable cells and a stem cell preparation lacking viable cells to said mammal,
wherein the size of said wound is reduced, and wherein the stem cell preparation is selected from the group consisting of a mesenchymal stem cell (MSC) preparation obtained from fat tissue or bone marrow, a neural stem cell (NSC) preparation, an umbilical cord blood stem cell preparation, an embryonic stem cell preparation, and an induced pluripotent stem cell preparation.

2. The method of claim 1, wherein said mammal is a human.

3. The method of claim 1, wherein said wound is selected from the group consisting of an abrasion, incision, laceration, puncture, avulsion, and burn.

4. The method of claim 1, wherein said wound is a slow healing wound or a scar.

5. The method of claim 1, wherein the size of said wound is reduced by at least 5%.

6. The method of claim 1, wherein said transdermally administering comprises iontophoresis.

7. The method of claim 6, wherein said iontophoresis comprises from about 0.1 mA/cm2 to about 1.0 mA/cm2.

8. The method of claim 1, wherein said transdermally administering comprises microinjection.

9. The method of claim 8, wherein said microinjection comprises using a microneedle array.

10. The method of claim 1, wherein said transdermally administering comprises a transdermal delivery device.

11. The method of claim 10, wherein said transdermal delivery device is an adhesive patch.

12. The method of claim 1, wherein said amnion tissue preparation comprises an amnion tissue preparation prepared from about 1 mg to about 10 g of amnion tissue.

13. (canceled)

14. The method of claim 1, wherein said amnion tissue preparation is a human amnion tissue preparation.

15. The method of claim 13, wherein said stem cell preparation is a human mesenchymal stem cell preparation obtained from fat tissue or bone marrow.

16. A method for treating a skin disorder in a mammal, wherein said method comprises:
transdermally administering a composition comprising an amnion tissue preparation lacking viable cells and a stem cell preparation lacking viable cells to said mammal,
wherein a symptom of said skin disorder is reduced, and wherein the stem cell preparation is selected from the group consisting of a mesenchymal stem cell (MSC) preparation obtained from fat tissue or bone marrow, a neural stem cell (NSC) preparation, an umbilical cord blood stem cell preparation, an embryonic stem cell preparation, and an induced pluripotent stem cell preparation.

17. The method of claim 16, wherein said mammal is a human.

18. The method of claim 16, wherein said skin disorder is selected from the group consisting of eczema, psoriasis, rosacea, seborrheic dermatitis, acne, cold sores, shingles, basal cell carcinoma, squamous cell carcinoma, melanoma, actinic keratosis, vitiligo, melasma, and lentigenes.

19. The method of claim 16, wherein said skin disorder is psoriasis.

20. The method of claim 16, wherein said symptom is eliminated.

21. The method of claim 16, wherein said transdermally administering comprises iontophoresis.

22. The method of claim 21, wherein said iontophoresis comprises from about 0.1 mA/cm2 to about 1.0 mA/cm2.

23. The method of claim 16, wherein said transdermally administering comprises microinjection.

24. The method of claim 23, wherein said microinjection comprises using a microneedle array.

25. The method of claim 16, wherein said transdermally administering comprises a transdermal delivery device.

26. The method of claim 25, wherein said transdermal delivery device is an adhesive patch.

27. The method of claim 16, wherein said amnion tissue preparation comprises an amnion tissue preparation prepared from about 1 mg to about 10 g of amnion tissue.

28. (canceled)

29. The method of claim 16, wherein said amnion tissue preparation is a human amnion tissue preparation.

30. The method of claim 28, wherein said stem cell preparation is a human mesenchymal stem cell preparation obtained from fat tissue or bone marrow.

31. A method for healing a wound in a mammal, wherein said method comprises:
transdermally administering a composition comprising an amnion tissue preparation lacking viable cells and a stem cell preparation lacking viable cells to said mammal,
wherein the size of said wound is reduced, and wherein the stem cell preparation lacking viable cells is made from about 0.3 million to about 3 million stem cells.

32. The method of claim 1, wherein the composition lacks viable cells.

33. The method of claim 16, wherein the composition lacks viable cells.
